(19) United States
(12) Patent Application Publication
(10) Pub. No.: US 2024/0093279 A1
(43) Pub. Date: Mar. 21, 2024

(54) ANTIBODY AND DNA OLIGO CONJUGATES AND THEIR APPLICATION IN ELISA-PCR TESTS

(71) Applicant: SUREDX LLC, Carlsbad, CA (US)

(72) Inventors: Claire Tianen MENG, San Marcos, CA (US); Allan Haotian FANG, San Diego, CA (US); Yan ZHOU, San Diego, CA (US)

(21) Appl. No.: 18/467,085

(22) Filed: Sep. 14, 2023

Related U.S. Application Data
(60) Provisional application No. 63/407,835, filed on Sep. 19, 2022.

(51) Int. Cl. C12Q 1/6848; C12Q 1/70; G01N 33/53; G01N 33/532; G01N 33/569

(52) U.S. Cl. CPC C12Q 1/6848; C12Q 1/70; G01N 33/5306; G01N 33/532; G01N 33/56983; G01N 2333/4712

(57) ABSTRACT

An antibody-DNA conjugate set for detection of a target protein, comprising a first antibody-DNA conjugate having a first antibody which is capable of binding to the target protein; and a first DNA oligo linked to the first antibody; a second antibody-DNA conjugate having a second antibody which is capable of binding to the target protein; and a second DNA oligo linked to the second antibody.

### CROSS-REFERENCE TO RELATED PATENT APPLICATIONS

This application claims priority to and benefit of U.S. Provisional Application Ser. No. 63/407,835 which is filed on Sep. 19, 2022, and hereby incorporated by reference in its entirety.

### STATEMENT REGARDING THE SEQUENCE LISTINGS

The Sequence Listings associated with this application are provided in xml format in lieu of a paper copy and are hereby incorporated by reference into the specification. The name of the xml file containing the Sequence Listings is 1009984.101US2_NPA_SL.xml. The xml file is about 72 KB, was created on Sep. 13, 2023.

### FIELD OF THE INVENTION

The present invention relates generally to the field of molecular assays. More particular, the present invention relates to antibody and DNA oligo conjugates, and their use in detection of a target protein via an ELISA-PCR test platform.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Traditional immunoassay and molecular diagnosis are two major testing methods for detection of a target molecule (e.g. a target protein, DNA or RNA) in the field of medical diagnosis. Immunoassay is a selective bioanalytical method that measures the presence or concentration of a target protein (e.g. an enzymatic protein in a subject, or a capsid protein of a virus) through the use of an antibody or an antigen as a biorecognition agent. For decades, immunoassay has played an important role in medical diagnosis. However, since the 1990s, the molecular diagnosis, e.g. PCR test, has gradually replaced the central position of the immunoassays, due to its high sensitivity, high specificity, and its capability for quantitative analysis. Despite the rise of molecular diagnosis, immunoassays remain irreplaceable due to its unique advantages in protein-based detection, as well as its simple operation and low cost, as compared to the molecular diagnosis.

Molecular diagnosis, on the other hand, is undergoing exponential growth in fields like infectious diseases. For example, in 2018, the global infectious disease diagnosis market was USD 22.4 billion. Since 2020, COVID 19 has had a huge economic and social impact in the world. In the United States alone, the economic impact is estimated to be over US$16 trillion. However, shortcomings of the molecular diagnosis are attracting unprecedented attention during this time. Higher and better sensitivity and specificity are long-felt need and yet unmet, as both false negatives and false positives have serious clinical and social consequences. Minimal human intervention for the molecular diagnosis is also desired, as manual operation is a main factor that contributes to the errors in results. The speed of the molecular tests also needs to be improved, as immediate detection results are of great significance in epidemic control due to the contiguous nature of the COVID 19. As such, the existing mainstream diagnostic technology has been challenged unprecedentedly. For example, the PCR, which is the mainstream molecular diagnostic technology, is exposed for its low sensitivity, lab facility/training requirements, long waiting time for results, and a series of other shortcomings.

Currently, in the market of molecular diagnosis, rapid diagnosis is highly desirable (time from sample to result is less than 60 minutes), on the basis of ensuring the characteristics of high specificity and sensitivity of the test. Testing flexibility should also be improved such that testing of single samples or small batches of samples can be conducted without waiting for a large batch. Moreover, fully automatic molecular diagnostic instruments are still limited worldwide, which limits the application of molecular diagnostics. Multiplex detection is needed, because the timely diagnosis of various infectious diseases requires simultaneous rapid inspection of multiple pathogens. In addition, transportation and storage need to be simplified because the reagents used in molecular diagnostics need to be transported in the cold chain, which increases the overall cost of tests.

Therefore, heretofore unaddressed needs exist in the art to address the aforementioned deficiencies and inadequacies.

### SUMMARY OF THE INVENTION

In light of the foregoing, this invention discloses an ELISA-PCR test, which combines the high specificity of the enzyme-linked reaction and the amplification of PCR in a breakthrough, high-sensitivity protein quantitative analysis.

In one aspect of the invention, an antibody-DNA conjugate set for detection of a target protein comprises a first antibody-DNA conjugate having a first antibody which is configured to bind to the target protein; and a first DNA oligo linked to the first antibody; and a second antibody-DNA conjugate having a second antibody which is configured to bind to the target protein; and a second DNA oligo linked to the second antibody.

In one embodiment, the first DNA oligo is covalently linked to the first antibody, and the second DNA oligo is covalently linked to the second antibody.

In one embodiment, the first DNA oligo is covalently linked to the first antibody on its 3′ end, and the second DNA oligo is covalently linked to the second antibody on its 5′ end.

In one embodiment, the first DNA oligo and second DNA oligo each has a length between 20-300 nucleotide bases.

In one embodiment, the first antibody and second antibody each binds to a different site of the target protein.

In one embodiment, when the first antibody and second antibody each binds to the target protein, the 5′ end of the first DNA oligo and the 3′ end of the second DNA oligo attaches to each other.

In one embodiment, the first antibody and second antibody, the first DNA oligo and second DNA oligo, and the target protein form a close loop structure.

In one embodiment, the target protein is selected from the group consisting of marker proteins of pathogens, marker proteins of infectious diseases; marker proteins of genetic diseases, marker proteins of acquired diseases.

In one embodiment, the target protein is a nucleocapsid protein of SARS-Cov2 virus.

In one embodiment, the target protein is Troponin I, AZ biomarkers, and cancer biomarkers.

In another aspect of the invention, a method for quantitatively detecting a target protein with a pair of antibody-DNA conjugates comprises adding a first antibody-DNA conjugate to a sample having the target protein; and, adding a second antibody-DNA conjugate to the sample having the target protein.

In one embodiment, the first antibody-DNA conjugate having a first antibody which is configured to bind to the target protein and a first DNA oligo linked to the first antibody; the second antibody-DNA conjugate having a second antibody which is configured to bind to the target protein; and a second DNA oligo linked to the second antibody.

In one embodiment, the first DNA oligo is covalently linked to the first antibody on its 3′ end, and the second DNA oligo is covalently linked to the second antibody on its 5′ end.

In one embodiment, the first antibody and second antibody each binds to the target protein, the 5′ end of the first DNA oligo and the 3′ end of the second DNA oligo attaches to each other.

In one embodiment, the method further comprises adding a first primer and a second primer to the sample, wherein the first primer is complementary to a sequence at the 3′ end of the first DNA oligo, and the second primer is identical to a sequence at the 5′ end of the second DNA oligo.

In one embodiment, the method further comprises starting PCR cycle so as to amplify the first and second DNA oligos with the first and second primers.

In one embodiment, the method further comprises quantitatively analyzing the result of the PCR amplification so as to determine the amount of the target protein in the sample.

In one embodiment, the target protein is selected from the group consisting of marker proteins of pathogens, marker proteins of infectious diseases; marker proteins of genetic diseases, marker proteins of acquired diseases.

In one embodiment, the target protein is a nucleocapsid protein of SARS-Cov2 virus.

In one embodiment, the target protein is Troponin I.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 provides a systematic illustration of the ELISA-PCR test in which an antibody-DNA conjugate detects a target protein.

FIG. 2A-2B illustrates quantitative detection results for SARS-Cov2 virus using antibody-DNA oligo conjugates in the ELISA-PCR test.

FIG. 3A-3B provides a comparison of test sensitivity between traditional ELISA test and ELISA-PCR test SARS-Cov2 virus using antibody-DNA conjugates.

FIG. 4A-4B provides a comparison of test range between traditional ELISA test and ELISA-PCR test using antibody-DNA conjugates.

FIG. 5 illustrates testing of troponin I using antibody-DNA conjugates with the ELISA-PCR test.

FIG. 6 illustrates ELISA-PCR test result for detection of human cardiac troponin T (cTnT) reaches a LOD of 0.5 pg/ml.

FIG. 7 illustrates testing of Tau protein using antibody-DNA conjugates with the ELISA-PCR test.

FIG. 8A illustrates an embodiment of a test plate to be used for an integrated device for performing the ELISA-PCR test.

FIG. 8B illustrates an embodiment to be used for an integrated device for performing the ELISA-PCR test.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the terms “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as thee-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used in the disclosure, the terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

As used in the disclosure, the term “oligonucleotides (oligonucleotide)” or “oligonucleotides (oligo)” refer to short nucleotide sequences.

As used in the disclosure, nucleic acid (for example, DNA) molecule is referred to as having “5′ end” and “3′ end”, because mononucleotide is with such side Formula is reacted to produce oligonucleotides or polynucleotides: described mode makes 5′ phosphoric acid of a mononucleotide pentose ring with a side To the 3′ oxygen being attached to its ortho position mononucleotide pentose ring via phosphodiester bond. Therefore, the end of oligonucleotides or polynucleotides End, if its 5′ phosphoric acid is not connected with 3′ oxygen of mononucleotide pentose ring, be then referred to as “5′ end”, and if its 3′ oxygen not with 5′ phosphoric acid of mononucleotide pentose ring subsequently connect, then be referred to as “3′ end”. As used herein, nucleotide sequence, even if Inside at bigger oligonucleotides or polynucleotides, it is possible to be referred to as that there are 5′ and 3′ ends. At linear or ring-shaped DNA molecule In, the discrete element is referred to as “upstream” or the 5′ ends of “downstream” or 3′ end element. This term reflects transcribes with 5′ ends to 3′ end mode along DNA carrying out the fact. The promoter transcribed of the gene connecting and enhancer element is instructed to be usually located at 5′ ends of the code area or upstream. But, enhancer element even also can be sent out when being positioned at the 3′ end of the promoter element and code area Wave its effect. Transcription termination and polyadenylation signals are positioned at 3′ ends or the downstream of the coding area.

As used in the disclosure, the term “conjugate” is intended to refer to the entity formed as a result of covalent or non-covalent attachment or linkage of an antibody or other molecule, e.g., a DNA oligo attached to an antibody. One example of a conjugate is an “antibody-DNA conjugates”, that is, an antibody covalently linked to at least one DNA oligo of 20-300 bases. As used in the disclosure, the terms “function” and “functional” and the like refer to a biological, enzymatic, or therapeutic function.

As used in the disclosure, “homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

As used in the disclosure, “isolated” refers to material that is substantially or essentially free from components that normally accompany it in its native state. For example, an “isolated peptide” or an “isolated polypeptide”, “isolated DNA” and the like, as used herein, includes the in vitro isolation and/or purification of a peptide, polypeptide, DNA molecule from its natural cellular environment, and from association with other components of the cell; i.e., it is not significantly associated with in vivo substances.

The term “linkage,” “linker,” “linker moiety,” or “L” is used herein to refer to a linker that can be used to separate an antibody or a fragment of the antibody from DNA, or to separate a first agent from another agent, for instance where two or more agents are linked to form an antibody-DNA conjugate. The linker may be physiologically stable or may include a releasable linker such as an enzymatically degradable linker (e.g., proteolytically cleavable linkers). In certain aspects, the linker may be a peptide linker, for instance, as part of an antibody protein. In some aspects, the linker may be a non-peptide linker or non-proteinaceous linker, e.g. a DNA. In some aspects, the linker may be particle, such as a nanoparticle.

As used in the disclosure, the terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between.

As used in the disclosure, in certain embodiments, the “purity” of any given agent in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used in the disclosure, term “sample” and “samples” are used with its broadest sense herein, and can include biological sample and ring Border sample. Patient Sample A can include the sample of all types obtained from mankind and other animals, and including but not limited to, body fluid is all such as urine, blood (whole blood, serum and plasma), fecal matter, cerebrospinal fluid (CSF), seminal fluid and saliva and solid tissue. These biological samples can also be material originate from patient but further developed in vitro, such as cell culture and tissue culture. Biological samples can be animal, including the mankind, fluid or tissue.

As used in the disclosure, the term “thermal cycler” refers to a programmable thermal cycler instrument, such as used for performing PCR Device.

As used in the disclosure, the term “amplifying reagent” can refer to those reagents (the such as DNA required for amplification of nucleic acid sequences Polymerase, deoxyribonucleoside triphosphate, buffer solution etc.). A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to: carboxylate ester, phosphate ester, anhydride, acetal, ketal, acyloxy alkyl ether, imine, orthoester, thio ester, thiol ester, carbonate, and hydrazone, peptides and oligonucleotides.

As used in the disclosure, the term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

As used in the disclosure, the terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

As used in the disclosure, terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length.

As used in the disclosure, by “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis is true. If the obtained p-value is smaller than the significance level, then the null hypothesis is rejected. In simple cases, the significance level is defined at a p-value of 0.05 or less.

As used in the disclosure, the term “complementary” is used for mentioning by base pairing rules related to many nucleotides (that is, nucleotide sequence). For example, for sequence “A-G-T”, complementary with sequence “T-C-A”. Complementarity can be “portion point”, some in the base of its amplifying nucleic acid are mated according to base pairing rules. Or, can exist between nucleic acids “completely (complete)” or “(total) just thoroughly” complementary.

As used in the disclosure, a “subject,” as used herein, includes any animal that exhibits a symptom, or is at risk for exhibiting a symptom, which can be diagnosed with an antibody-DNA oligo conjugate of the invention. Suitable subjects (patients) include laboratory animals (such as mouse, rat, rabbit, or guinea pig), farm animals, and domestic animals or pets (such as a cat or dog). Non-human primates and, preferably, human patients, are included.

As used in the disclosure, “substantially” or “essentially” means nearly totally or completely, for instance, 95%, 96%, 97%, 98%, 99% or greater of some given quantity.

As used in the disclosure, “substantially free” refers to the nearly complete or complete absence of a given quantity for instance, less than about 10%, 5%, 4%, 3%, 2%, 1%, 0.5% or less of some given quantity. For example, certain compositions may be “substantially free” of cell proteins, membranes, nucleic acids, endotoxins, or other contaminants.

As used in the disclosure, the term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

As used in the disclosure, the terms “real-time” and “real-time continuous” are interchangeable and refer to a method in which data collection occurs during the course of a polymerization reaction by periodic monitoring. Thus, the method combines amplification and detection into a single step.

As used in the disclosure, the term “quantitative PCR” or “qPCR” refers to the use of the Polymerase Chain Reaction (PCR) to quantify target DNA.

As used in the disclosure, the term “Ct” and “cycle threshold” refer to the time at which the fluorescence intensity is greater than background fluorescence. Characterized by the time point (or PCR cycle) at which the amplification of the target is first detected. Thus, the greater the amount of target DNA in the starting material, the faster a significant increase in fluorescence signal will occur, resulting in a lower Ct.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

The current invention introduces an ELISA-PCR test, which combines the high specificity of the enzyme-linked immunoassay reactions with the amplification of PCR in a breakthrough, high-sensitivity protein quantitative analysis.

In one aspect of the invention, an antibody-DNA conjugate set for detection of a target protein, comprising a first antibody-DNA conjugate having a first antibody which is capable of binding to the target protein; and a first DNA oligo linked to the first antibody; and a second antibody-DNA conjugate having a second antibody which is capable of binding to the target protein; and a second DNA oligo linked to the second antibody.

In one embodiment of the invention, the first DNA oligo is covalently linked to the first antibody on its 3′ end, and the second DNA oligo is covalently linked to the second antibody on its 5′ end.

In one embodiment of the invention, the first and second antibody each binds to the target protein, the 5′ end of the first DNA oligo and the 3′ end of the second DNA oligo attaches to each other.

In one embodiment of the invention, the method further comprising a step of adding a primer pair to the sample, wherein approximately half of the primer complementarity binds to the 5′ end of the first DNA oligo, and approximately the other half of the primer binds to the 3′ end of the second DNA oligo.

In one embodiment of the invention, the method further comprises a step of starting PCR cycle so as to amplify the first and second DNA oligos with the primer.

In one embodiment of the invention, the method further comprises a step of quantitatively analyzing the result of the PCR amplification so as to determine the amount of the target protein in the sample.

In one embodiment of the invention, the target protein is selected from a group comprising marker proteins of pathogens, marker proteins of infectious diseases; marker proteins of genetic diseases, marker proteins of acquired diseases.

In one embodiment of the invention, the target protein is a nucleocapsid protein of SARS-Cov2 virus.

In one embodiment of the invention, the target protein is human Troponin I (cTnI).

In one embodiment of the invention, the target protein is human Troponin T (cTnT).

In one embodiment of the invention, the target protein is human Tau and human Tau phosphorylated at site of Thr18 (Tau phospho-Thr181 or Tau Thr181).

These and other aspects of the present invention are further described below. Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Antibody-DNA Conjugates

This invention provides a methodology to covalently label antibodies with DNA molecules of 20-300 bases in a site-specific way for downstream applications, such as Immuno-PCR, Proximity Ligation PCR or any immuno-assays using antibody-DNA conjugates as probes.

In one embodiment of the invention, these antibody-DNA conjugates have 1 to 10 DNA molecules per antibody. In another embodiment of the invention, more than 1 DNA molecules may be attached to an antibody. The DNA molecules are covalently attached to the heavy chain of the antibody. This labeling method does not interfere with the epitope binding site of the antibody so as to ensure the antibody binding affinity and specificity. This labeling method generates covalently labeled antibodies used in ELISA-PCR test. Comparing to traditional ELISA test, the ELISA-PCR test using the antibody-DNA conjugates increases the detection sensitivity of a target protein by 10 times or more.

In one embodiment of the invention, the antibody-DNA conjugates used in the ELISA-PCR test is produced according to the following steps:
(1) Modify a DNA oligo with one or more chemicals to form a DNA oligo with an active functional group. In one embodiment of the invention, during this reaction, the DNA oligo molecule is attached with an —NH₂ functional group at the 3′ or the 5′ end. The DNA-NH₂ is then incubated with DBCO, TCO, Tetrazines, Azide, Alkynes, or similar chemicals for 1-24 hours at room temperature (RT) around 25° C. at a ratio of 1:10.
(2) Modify antibody using click chemistry by adding 1-10 azide groups to the heavy chains of the DNA molecule. This process involves modifying the antibody heavy chain through an enzymatic step. For example, 1-10 microgram of Gal-T transferase was used per reaction with 100-10,000 microgram UDG-GalNaz to incubate with 100 to 1000 microgram antibody at approximate 30° C. overnight (>=18 hours).
(3) Incubate the DNA oligo(s) from step (1) with the activated antibody from step (2) to form the final product: a site-specific, covalently labeled antibody with 1-10 DNA oligo(s). In one embodiment, the 3′ or the 5′ end of the DNA oligo(s) with the —NH₂ functional group is/are attached to the carbohydrate group(s) of the antibody which is activated.

In one example of the invention, the antibody may be a Tau antibody, e.g. sc-21796 (clone Tau-13) which specifically binds to protein Tau, and is used as a marker of Alzheimer's disease. In one example of the invention, one or more DNA oligos are covalently linked to the antibody Tau antibody sc-21796 (clone Tau-13).

It should be noted that the antibody Tau antibody sc-21796 is only used as an example. Other antibodies, either commercially available or lab-generated, can be labeled via functional groups and thereafter covalently linked with DNA oligos for being used in the ELISA-PCR test to detect different target proteins. In certain embodiments, the antibody is selected from one or more of Tau antibodies on the market such as of Tau Thr181 specific antibody (PA5114656, Thermo Fisher), and Tau monoclonal antibody BT2 (Catalog #MN1010, Thermo Fisher), and etc.

In other embodiment, antibodies binding to virus, bacteria, diseases markers can be used for the ELISA-PCR test.

In one embodiment, a first DNA oligo may comprise one of SEQ ID Nos: 1-20, and a second DNA oligo may comprise one of SEQ ID No: 21-40. In particular, SEQ ID No: 1 and SEQ ID No. 21 form a pair of DNA Oligos. SEQ ID No: 2 and SEQ ID No. 22 form a pair of DNA Oligos. SEQ ID No: 3 and SEQ ID No. 23 form a pair of DNA Oligos. SEQ ID No: 4 and SEQ ID No. 24 form a pair of DNA Oligos. SEQ ID No: 5 and SEQ ID No. 25 form a pair of DNA Oligos. SEQ ID No: 6 and SEQ ID No. 26 form a pair of DNA Oligos. SEQ ID No: 7 and SEQ ID No. 27 form a pair of DNA Oligos. SEQ ID No: 8 and SEQ ID No. 28 form a pair of DNA Oligos. SEQ ID No: 9 and SEQ ID No. 29 form a pair of DNA Oligos. SEQ ID No: 10 and SEQ ID No. 30 form a pair of DNA Oligos. SEQ ID No: 11 and SEQ ID No. 31 form a pair of DNA Oligos. SEQ ID No: 12 and SEQ ID No. 32 form a pair of DNA Oligos. SEQ ID No: 13 and SEQ ID No. 33 form a pair of DNA Oligos. SEQ ID No: 14 and SEQ ID No. 34 form a pair of DNA Oligos. SEQ ID No: 15 and SEQ ID No. 35 form a pair of DNA Oligos. SEQ ID No: 16 and SEQ ID No. 36 form a pair of DNA Oligos. SEQ ID No: 17 and SEQ ID No. 37 form a pair of DNA Oligos. SEQ ID No: 18 and SEQ ID No. 38 form a pair of DNA Oligos. SEQ ID No: 19 and SEQ ID No. 39 form a pair of DNA Oligos. SEQ ID No: 20 and SEQ ID No. 40 form a pair of DNA Oligos.

In other embodiment, DNA oligos have other nucleotide sequences can be used.

### Process of ELISA-PCR Test

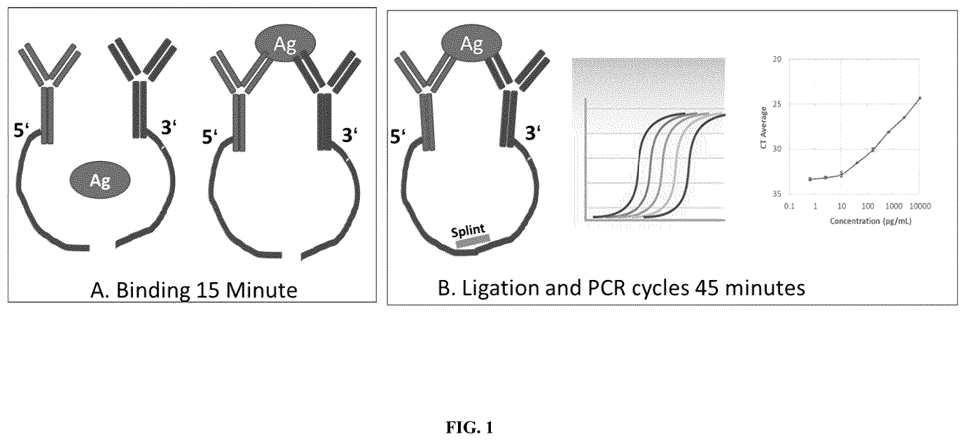

As illustrated in FIG. 1, an ELISA-PCR test has stage (A) and stage (B).

In one embodiment of the invention, during the stage (A), an ELISA-PCR test for detection of a target protein requires two different antibody-DNA conjugates. A first antibody-DNA conjugate binds to a specific region of the target protein, and the 3′ end of the first DNA oligo is covalently linked to the first antibody, while a second antibody-DNA conjugate binds to a different region of the target protein and the 5′ end of the second DNA oligo is covalently linked to the second antibody.

It should be noted that the first and second antibodies may bind to two separate binding sites of the target protein. Regardless of the binding site, the first and the second DNA oligos should have a length enough for the 5′ end of the first DNA oligo and the 3′ end of the second DNA oligo to be attached to each other.

During the stage (B) of the ELISA-PCR test, the 5′ end of the first DNA oligo and the 3′ end of the second DNA oligo are ligated to become one oligo. Once the first and second antibodies bind to the target protein, and the 5′ end of the first DNA oligo and the 3′ end of the second DNA oligo will be close enough to be ligated. After the ligation of the two DNA oligos, the target protein, the first and second antibodies, the first and second DNA oligos form a closed loop structure. During the stage (B), the solution contains a primer pair which are specific for the two DNA oligos. One primer of the primer pair is complementary and bind to the sequence having about 1-50 based pair at the 3′ end of the first DNA oligo. In one embodiment, the range for the base pair is about 10-40 bp. In one embodiment, the range for the base pair is about 15-35 bp. In one embodiment, the range for the base pair is about 20-30 bp. In one embodiment, the range for the base pair is about 18-25 bp. Following synthesis and at the end of the first cycle, each double-stranded DNA molecule consists of one new and one old DNA strand. The other primer of the primer pair is identical to the first 5-40 bases of the 5′ end of the second DNA oligo and will bind to the 3′ end of the newly synthesized DNA after the first cycle. PCR then continues with additional cycles that repeat the aforementioned steps. The newly synthesized DNA segments serve as templates in later cycles, which allow the DNA target to be exponentially amplified millions of times.

As an example, the primer comprises the nucleotide sequences of SEQ ID No: 41-60 and/or 61-80. In particular, the primer pair has SEQ ID Nos. 41 and 61 are used for amplification of DNA conjugates having DNA Oligos of SEQ ID Nos: 1 and 21. Other examples are presented in Table 3.

A regular real time PCR test is then performed. If the first and second antibodies both bind to the target protein, and the 5′ end of the first DNA oligo and the 3′ end of the second DNA oligo are ligated, the PCR test would produce amplification DNA sequences, starting with the primers.

Because all DNA polymerases possess 5′—>3′ polymerase activity, which is the incorporation of nucleotides to extend primers at their 3′ ends in the 5′ to 3′ direction, the second DNA oligo whose 3′ end is attached to the second antibody should be amplified first and most. The amount of the first synthesis DNA molecules is correlated with the presence of the target protein. The more of the target protein presents, the quicker the PCR reaction will reach the detectable threshold, the Ct or Cq value of real-time PCR.

In one embodiment of the invention, both primers have 5-40 bases. One having ordinary skill in the art can design primers with different sequences according to the first and second DNA oligo sequences. That is, approximately half of the primers should be complementary and binding to the sequence at the 3′ end of the second DNA oligo, while the other half of the primers should be identical to the 5′ end of the first DNA oligo and will bind to the 3′ end of the newly synthesis DNA molecules.

Amplification of the loop structure with the primers reflects the existence of the target protein.

### Advantages of ELISA-PCR

Compare to the traditional ELISA test, the ELISA-PCR test of the present invention has a number of advantages.

First, the ELISA-PCR test has a higher sensitivity than the traditional ELISA test. In one embodiment of the invention, the ELISA-PCR test can detect the target proteins with 10-100 times of the sensitivity as compared to traditional enzyme-linked reactions.

The ELISA-PCR test also has a higher specificity as compared to the traditional ELISA test, because there are two antibodies to bind to the target protein at the same time to form a closed DNA loop, so as to render subsequent nucleic acid amplification possible.

In light of foregoing, in one embodiment, the upper and lower detection limits for a target protein span more than 5 logs. Also, the ELISA-PCR test significantly reduces the necessary amount of the sample for processing. In one embodiment, less than 1 μL of sample is required. In one embodiment, less than 5 μL of sample is required. In another embodiment, 5-10 μL of sample is required. In another embodiment, 10-20 μL of sample is required.

Moreover, by using different DNA oligos and primers, more than one target protein can be detected. In one embodiment, a first antibody with a first DNA oligo and a second antibody with a second oligo bind to a first target protein such that the PCR amplification would be started with a first primer pair. Simultaneously, a third antibody with a third DNA oligo and a fourth antibody with a fourth oligo bind to a second target protein such that PCR amplification would be started with a second primer pair. By analyzing the product produced during the PCR amplification, a skilled artisan would determine the amount of both the first and second target proteins in the sample.

In one embodiment, the ELISA-PCR test can be used to quantitatively measure at least two target proteins. In one embodiment, the ELISA-PCR test can be used to quantitatively measure at least five target proteins. In one embodiment, the ELISA-PCR test can be used to quantitatively measure at least ten target proteins.

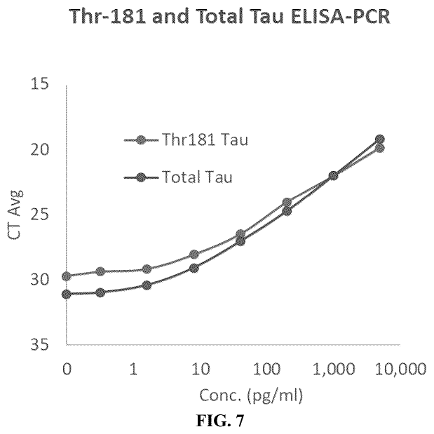

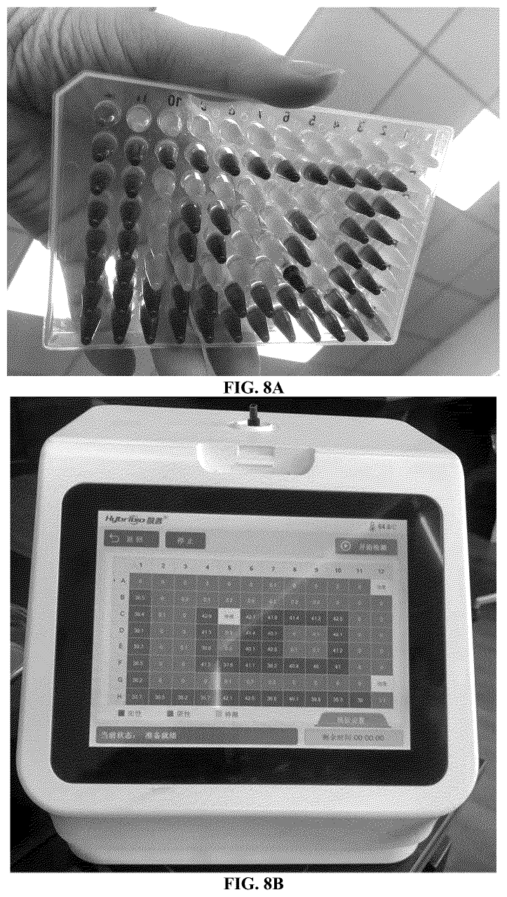

Moreover, the steps of the ELISA-PCR test are simple such that the sample collected from an individual does not require pre-treatment, elution, and/or dilution. In one embodiment of the invention, the operation of the ELISA-PCR test can be fully automatic, integrated with automatic data analysis. In one embodiment of the invention, the automatic operation of the ELISA-PCR test can be performed by a miniaturized instrument as illustrated in FIGS. 7A and 7B.

In one embodiment, a typical test protocol is provided as below in Table 1.

TABLE 1
Protocol for ELISA-PCR test.

| | |
|---|---|
| Step 1: | Antigen Standard Curve Preparation |
| 1a | Reconstitute Protein standard with Assay Dilution Buffer |
| 1b | Prepare standard 1 (5000 pg/mL) from reconstituted protein standard |
| 1c | Make serial 1:5 dilution for standard 2-7 in Assay Dilution Buffer, standard 8 |
| | is blank. |
| Step 2: | Preparation of Pair for Verification |
| | Record antibody conjugate A and B clone numbers for the pairs to be verified |
| | in Table 4 in Summary Report tab |
| 2a | Prepare each antibody pair by diluting Antibody Conjugate A and Antibody |
| | Conjugate B in Antibody Conjugate Dilution Buffer (1 to 60) |
| 2b | Aliquote each diluted antibody pair from step 2a into 4 wells (42 uL in each) |
| Step 3: | PLA Binding Plate Preparation and qPCR |
| 3a | To qPCR plate on a cold block, use multi-channel pipet, add 2 ul of Antigen |
| | standard 1-12 to the bottom of the wells in all rows |
| | To the same qPCR plate, use multi-channel pipet, add 2 ul of Antibody |
| | Conjugate A and B Pair mix to the wall of the wells in all columns. |
| | Seal plate and spin down. Incubate for 10 minutes to 1 hour at RT or overnight |
| | at 4 C. |
| 3b | Add 16 ul of PLA PCR master mix + Ligase to all reaction wells |
| | Run qPCR using Verification template. |
| Step 4: | Data Analysis |
| 4a | Once qPCR run is finished, export the raw data as .EDS file |
| 4b | Upload .EDS file to qPCR software for analysis |
| 4c | Standard curve plot, standard curve, and % bias will be generated. |

In one embodiment, antibodies specifically bind to a target protein of interest can be either lab-produced using techniques commonly known in the field, or obtained as commercial products.

The example antibodies are described in Table 2 below.

TABLE 2
Antibodies used in the conjugates of ELISA-PCR Test

| Pair# | Antigen | Vendor | Cat Number | Clone | Source |
|---|---|---|---|---|---|
| | Tau | R&D Systems | MAB106291 | 1032501 | mouse IgG |
| | | R&D Systems | MAB3494 | 376720 | mouse IgG |
| | Tau | Thermo Fisher | MN1050 | AT270 | mouse IgG |
| | Thr181 | R&D Systems | MAB3494 | 376720 | mouse IgG |
| | cTNI | Creative | CABT-L1129 | na | mouse IgG |
| | | Diagnostics | | | |
| | | Creative | CABT-L1130 | na | mouse IgG |
| | | Diagnostics | | | |
| | cTNT | Creative | CABT-L702 | TD75-14 | Rabbit IgG |
| | | Diagnostics | | | |
| | | Creative | DMAB1823MH | 2G3 | mouse IgG |
| | | Diagnostics | | | |

Other examples may include:
Tau monoclonal antibody MAB3494 (Clone 376720)
Tau Thr181 specific antibody MN1050, (Clone AT270),
Tau monoclonal antibody MAB106291 (Clone 1032501)
The examples of DNA oligos and primers are listed as below.

TABLE 3
Examples of DNA oligos and primers

| | Oligo A | Oligo B | Forward Primer | Reverse Primer |
|---|---|---|---|---|
| Example | aattggacgt ggctttcgcg | ccgcgcatcc | aattggacgt | cgtattccat |
| 1 | ttagtacgta gcatggtcac | tatgtatcaa gttagttcta | ggctttcgcg ttagt | tagaactaac ttgat |
| | acaagcacag tagatcctgc | atggaatacg (SEQ ID | (SEQ ID No. 41) | (SEQ ID No. 61) |
| | (SEQ ID No. 1) | No. 21) | | |
| Example | tcataaggag tccggtgtag | ggcggtaagg | tcataaggag | tgtcttaccg |
| 2 | cgaaagatca aggcgaccct | tatcactcaa | tccggtgtag cgaaa | agcctgcttc ttgag |
| | aggtagcaac cgccggctcc | gaagcaggct | (SEQ ID No. 42) | (SEQ ID No. 62) |
| | (SEQ ID No. 2) | cggtaagaca (SEQ ID | | |
| | | No. 22) | | |
| Example | cttccgctgg gatccaacgt | gtgactatct | cttccgctgg | gga tcggca |
| 3 | tggcggccga agccgccatt | gtgccagatc | gatccaacgt tggcg | gtctgccaga cgatct |
| | ccatagtgag tccttcgtct | gtctggcaga | (SEQ ID No. 43) | (SEQ ID No. 63) |
| | (SEQ ID No. 3) | ctgccgatcc (SEQ ID | | |
| | | No. 23) | | |
| Example | tcctagatac cgcactctgg | cctgacctaa | tcctagatac | cggcggagcc |
| 4 | gcagtacgag gtaatgccag | cggtaagagg | cgcactctgg gcagt | attatgtgag cctct |
| | tcacccagtg ccgaacaaca | ctcacataat | (SEQ ID No. 44) | (SEQ ID No. 64) |
| | (SEQ ID No. 4) | ggctccgccg (SEQ ID | | |
| | | No. 24) | | |
| Example | tccagttcgg tcagtgggtc | tagcgtgacg | tccagttcgg | tacgatcgca |
| 5 | actgcaagta gtcgattgca | gccgcagggg | tcagtgggtc actgc | ttttatgggt cccct |
| | ttgccaatct ccgagtgatt | acccataaaa | (SEQ ID No. 45) | (SEQ ID No. 65) |
| | (SEQ ID No. 5) | tgcgatcgta (SEQ ID | | |
| | | No. 25) | | |
| Example | aaaatcacca gtgcccaaga | gtaatgaata | aaaatcacca | tcgtccctct |
| 6 | ccaggggggc tcgccgcgtt | ttcagtagaa | gtgcccaaga ccagg | aacacagact ttcta |
| | ggctaatccc ggtacatctt | agtctgtgtt | (SEQ ID No. 46) | (SEQ ID No. 66) |
| | (SEQ ID No. 6) | agagggacga (SEQ ID | | |
| | | No. 26) | | |
| Example | cgagcggcgc agccgattag | tatcttcctg | cgagcggcgc | tagcaacagc |
| 7 | gaccatgtag aacatttgtt | cccagtggcg | agccgattag gacca | gataaccatc cgcca |
| | acaagacttc ttttaaacac | gatggttatc gctgttgcta | (SEQ ID No. 47) | (SEQ ID No. 67) |
| | (SEQ ID No. 7) | (SEQ ID No. 27) | | |
| Example | gcttagttca acctcgaata | gtgcggacgg | gcttagttca | cggtcagatt |
| 8 | cctcgtatca ttgtgcacct | cgttgcaact | acctcgaata cctcg | aggccctgga agttg |
| | gccggtcacc agccaacgat | tccagggcct | (SEQ ID No. 48) | (SEQ ID No. 68) |
| | (SEQ ID No. 8) | aatctgaccg (SEQ ID | | |
| | | No. 28) | | |
| Example | atgacatgtg gatgggcagt | gactagagtg | atgacatgtg | gcataacggg |
| 9 | ggccggttgt tacacgcctg | gcgagaacta | gatgggcagt ggccg | aggccctgg tagtt |
| | ccgcgacgct gaatgacccg | tggcgtgtga | (SEQ ID No. 49) | (SEQ ID No. 69) |
| | (SEQ ID No. 9) | cccgttatgc (SEQ ID | | |
| | | No. 29) | | |
| Example | cggtctagct gactgtctat | gccttaggat | cggtctagct | atctcgacca |
| 10 | cgcctaggtc aaatagggag | tcacttcagc | gactgtctat cgcct | ggccctggc gctga |
| | ctttgatatc tgcgtgtcca | gcgcaggcct | (SEQ ID No. 50) | (SEQ ID No. 70) |
| | (SEQ ID No. 10) | gggtcgagat (SEQ ID | | |
| | | No. 30) | | |
| Example | agtttatccc accaaactat | ggctcagctc tatttttgtg | agtttatccc | ggcggaccag |
| 11 | agccgtacag gccgaaatct | gtcatgggtt | accaaactat agccg | aggccctgg cacaa |
| | taagtcatat cgcgcgacta | ctggtccgcc (SEQ ID | (SEQ ID No. 51) | (SEQ ID No. 71) |
| | (SEQ ID No. 11) | No. 31) | | |
| Example | gcgtgcccag ggtatattag | cggaaacggg | gcgtgcccag | attttcgttt |
| 12 | gtcagcatcg gatggactga | tgcgtggact | ggtatattag gtcag | aggccctgg agtcc |
| | catgaacctt tacaccgaag | agcgaggagc | (SEQ ID No. 52) | (SEQ ID No. 72) |
| | (SEQ ID No. 12) | aaacgaaaat (SEQ ID | | |
| | | No. 32) | | |
| Example | gtccacccga ccgtacatag | cgtttgcatt | gtccacccga | ccttatgctc |
| 13 | aaatgagggt ccccgtacgc | taagggccgc | ccgtacatag aaatg | aggccctgg gcggc |
| | ccacgcacct gttcgctcgt | acgaaccaca | (SEQ ID No. 53) | (SEQ ID No. 73) |
| | (SEQ ID No. 13) | gagcataagg (SEQ ID | | |
| | | No. 33) | | |
| Example | gtcaccatgt accaagggcg | ggtttacgtg | gtcaccatgt | cctagcctgt |
| 14 | ataacgatcg gtgggagtat | ggaaaggtgc | accaagggcg ataac | aggccctgg gcacc |
| | tcatcgtggt gaagacgctg | ttgtgtccca | (SEQ ID No. 54) | (SEQ ID No. 74) |
| | (SEQ ID No. 14) | acaggctagg (SEQ ID | | |
| | | No. 34) | | |
| Example | gccggcgtgg aaggtaacag | gctgtccgat | gccggcgtgg | gccaacccca |
| 15 | caccgctgcg agcctaatgc | cgtatattag | aaggtaacag caccg | aggccctgg cctaat |
| | gccgtttcca cgaacacagg | gactccgcga | (SEQ ID No. 55) | (SEQ ID No. 75) |
| | (SEQ ID No. 15) | tggggttggc (SEQ ID | | |
| | | No. 35) | | |
| Example | tcctggcctg cttgatgtct | ggaagaggtc | tcctggcctg | ggagagtagc |
| 16 | cgtgaccttc ttagagatgg | gccctacaaa | cttgatgtct cgtga | aggccctgg tttgt |
| | acgaaatgtt tcgcgaccta | atagatttgc gctactctcc | (SEQ ID No. 56) | (SEQ ID No. 76) |
| | (SEQ ID No. 16) | (SEQ ID No. 36) | | |
| Example | aggacctcta gctcctttac | cgcaatgacg | aggacctcta | caggaaccga |
| 17 | aaagtgctgg ttccctttcc | gacgtattcc | gctcctttac aaagt | ggccctgga ggaat |
| | ggcgggatgc cttatctaaa | tctggccaca | (SEQ ID No. 57) | (SEQ ID No. 77) |
| | (SEQ ID No. 17) | tcggttcctg (SEQ ID | | |
| | | No. 37) | | |
| Example | gtataatgct ggagccctcc | caacgtgtcc | gtataatgct | ctcggccttg |
| 18 | cccaagcgtt cagggtgggg | gtgttcacgt | ggagccctcc cccaa | aggccctgg acgtg |
| | tttgctacga cttccgagtc | tatatgcgca | (SEQ ID No. 58) | (SEQ ID No. 78) |
| | (SEQ ID No. 18) | caaggccgag (SEQ ID | | |
| | | No. 38) | | |
| Example | aagtggcagc ctaaacgatg | gtgttccggc tgttatcctg | aagtggcagc | gcatgattga |
| 19 | ttgggggctc gcgatgcaca | catcggaacg | ctaaacgatg ttggg | aggccctgg cagga |
| | cgctctggta caatacatac | tcaatcatgc (SEQ ID | (SEQ ID No. 59) | (SEQ ID No. 79) |
| | (SEQ ID No. 19) | No. 39) | | |
| Example | caaaagcttt aaacgcgagt | ttccactgtg tttgtctcat | caaaagcttt | acgctttgcg |
| 20 | tcccgcccat aacctggacc | gtaggacggg | aaacgcgagt tcccg | aggccctgg atgag |
| | gaatgcggga tcatgcatcg | cgcaaagcgt (SEQ ID | (SEQ ID No. 60) | (SEQ ID No. 80) |
| | (SEQ ID No. 20) | No. 40) | | |

### Embodiment 1: ELISA-PCR Application—Infectious Diseases COVID 19

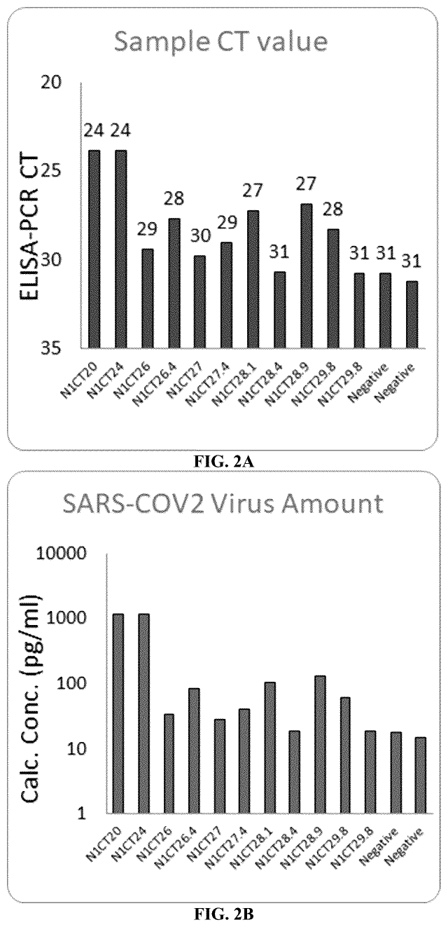

As illustrated in FIG. 2, the ELISA-PCR test of the present invention provides an accurate quantitative analysis of the SARS-Cov2 virus amount in eleven COVID-19 positive patients and two COVID-19 negative controls.

As reflected in FIG. 2, the ELISA-PCR test demonstrates an outstanding quantitative detection results for thirteen COVID-19 samples, especially for samples with low viral load. In one embodiment of the invention, the detection ranges from as low as 10 pg/ml to 1000 pg/ml.

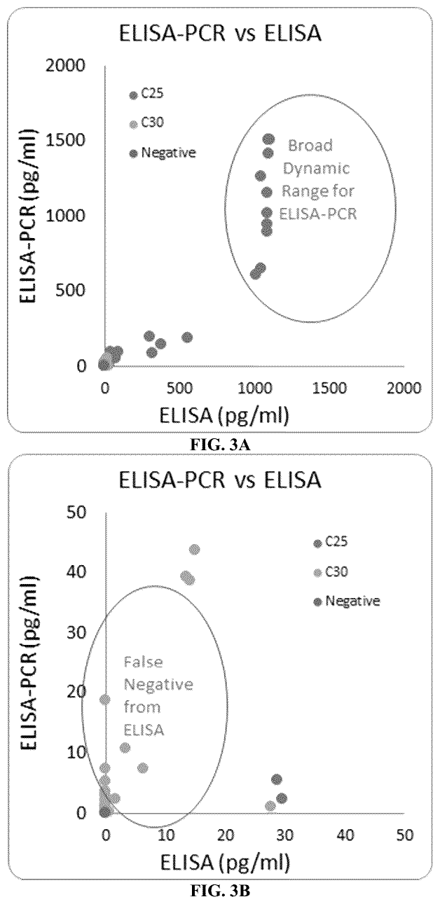

Moreover, as illustrated in FIG. 3, the present invention significantly reduces the false positive and false negative results which are common in traditional ELISA and other immunoassay test.

In particular, as shown in FIG. 3, detections of SARS-Cov2 virus are performed for 40 individual samples. The detected amounts of SARS-Cov2 virus are compared between the ELISA-PCR test and the traditional ELISA test.

As shown in FIG. 3, for samples having C25 high-load virus amount, according to panel (A), the results of the ELISA-PCR test and the traditional ELISA test are consistent, as shown in circled oval, which can be quantitatively analyzed within a higher detection limit. In particular, when the amount of SARS-Cov2 virus is between 500-1500 pg/ml, the results between the ELISA-PCR test and the traditional ELISA test are consistent. However, in the samples with low C30 virus load, the ELISA-PCR test can detect virus amount of the samples which cannot be correctly detected and has been identified as negative results (false negative) by the traditional ELISA.

In particular, as shown in FIG. 3, panel (B), when the SARS-Cov2 virus amount is under 30 pg/ml, the traditional ELISA test may fail to detect the existence of the SARS-Cov2 virus in the sample, and would report the sample as SARS-Cov2 negative. However, the ELISA-PCR test accurately detects the SARS-Cov2 virus amount which is under 30 pg/ml, under 20 pg/ml, under 10 pg/ml, or under 5 pg/ml.

Therefore, the ELISA-PCR test of the invention significantly improves the sensitivity and accuracy of the detection of SARS-Cov2 virus, and lowering the minimum amount of the virus which can be detected, as compared to the traditional ELISA test.

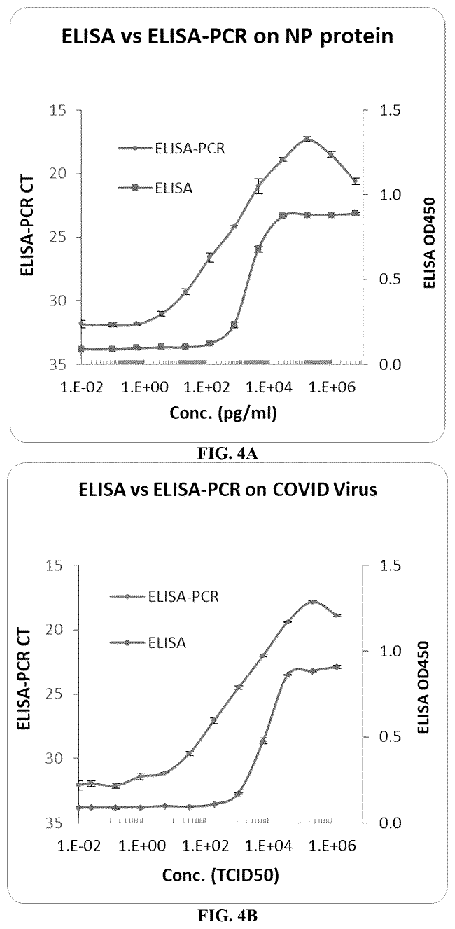

As illustrated in FIG. 4 and Table 4 below, the dynamic range for detection of the target protein is significantly broadened by the ELISA-PCR test of the present invention, as compare to the traditional ELISA test.

TABLE 4
Dynamic Range (log) for NP protein and COVID virus

| | | | |
|---|---|---|---|
| ELISA_NP (130-28000) | 2.33 | ELISA-PCR_NP (1-250,000) | 5.40 |
| ELISA_COVID (200-42000) | 2.32 | ELISA-PCR_COVID (1-167,000) | 5.22 |

As shown in FIG. 4, panels (A) and (B), the sensitivity and accuracy of the detection of the target protein NP and SARS-Cov2 are significantly broadened by the ELISA-PCR test. In particular, for both NP protein and SARS-Cov2 virus, when the concentration of the target protein is between 1 pg/ml and 10000 pg/ml, the limit of detection (LOD) detected by the ELISA-PCR is much lower (>10 fold) than that of the traditional ELISA test.

In one embodiment of the invention, pathogens can be detected includes COVID, MEMS, Influanza A and B, HIV, HPV, and etc.

### Embodiment 2: ELISA-PCR Application—Cardiovascular Diseases

The invention is also effective in detecting other target proteins typically used in medical diagnosis. In one embodiment of the invention, human troponin I and troponin T can be measured by the ELISA-PCR test of the current invention, so as to diagnose a medical condition related to the heart. Human troponin I and troponin T is a very specific myocardial enzyme, which is clinically used to diagnose acute myocardial infarction, heart failure etc. Using traditional immunoassays, detection of Troponin I and Troponin T within 3-4 hours after myocardial necrosis is feasible. However, with the more sensitive and faster ELISA-PCR test, the correct diagnosis and treatment can be performed earlier.

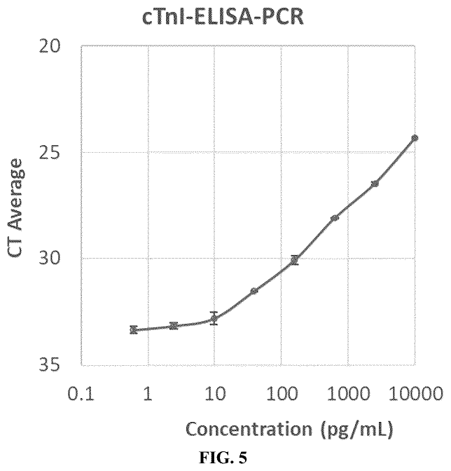

As shown in FIG. 5, the ELISA-PCR test for detection of human cardiac troponin I (cTnI) reaches a LOD of 2.4 pg/ml.

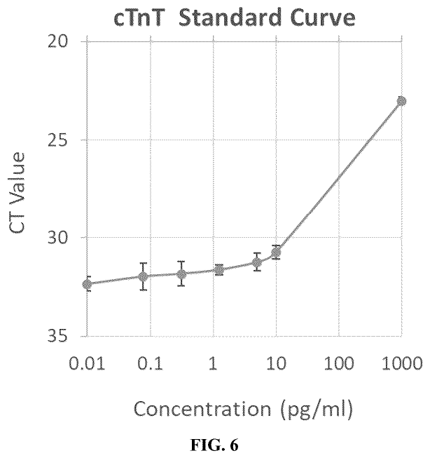

As shown in FIG. 6, the ELISA-PCR test for detection of human cardiac troponin T (cTnT) reaches a LOD of 0.5 pg/ml.

### Embodiment 3: ELISA-PCR Application—Neurodegenerative Diseases

In one embodiment of the invention, the ELISA-PCR test is used for detecting a target protein in certain neurodegenerative diseases. Tau protein plays a key role in certain neurodegenerative diseases such as Alzheimer's disease (AD). A significant amount of evidence shows that the first change in the brain occurs at least 15 years before AD symptoms start to develop and demonstrate in an AD patient. If the detection sensitivity can be improved, the correct diagnosis can be made significant earlier, as well as symptomatic treatment and prevention.

As shown in FIG. 7, using the ELISA-PCR test, the LOD of Total Tau is 1 pg/ml, and the LOD of Tau Thr181 is 2 pg/ml. As it can be seen, the LOD is significantly lower than existing testing methods, which can only detect Tau and Tau Thr181 at a concentration around 10-1000 pg/ml.

In a clinical test for detection of Tau 181 using the ELISA-PCR Test of the present invention, a total of 58 blood serum samples were collected from 58 AD patients. Among the 58 AD patients, 22 of the patents demonstrate mild recognition impairment. The detection of Tau 181 in these 58 AD patients are reflected in the Table 5 below.

TABLE 5
Detection of Tau 181 in 58 AD patients

| | Total Sample | Positive # | Positive % | |
|---|---|---|---|---|
| Normal | 27 | 6 | 78% | NPA* |
| Individual | | | | |
| AD | 58 | 43 | 74% | PPA* |
| Mild AD | 22 | 19 | 86% | |
| symptom | | | | |

*Positive Percent Agreement (PPA), also known as Sensitivity or True Positive Rate, measures the ability of a test or model to correctly identify positive cases among all actual positive cases. It is calculated using the formula PPA = (True Positives)/(True Positives + False Negatives). PPA represents the percentage of true positive cases correctly identified by the test or model out of all the actual positive cases.
Negative Percent Agreement (NPA), also known as Specificity or True Negative Rate, measures the ability of a test or model to correctly identify negative cases among all actual negative cases. It is calculated using the formula: NPA = (True Negatives)/(True Negatives + False Positives)
NPA represents the percentage of true negative cases correctly identified by the test or model out of all the actual negative cases.

In one embodiment of the invention, the target protein can be marker proteins of pathogens, marker proteins of infectious diseases; marker proteins of genetic diseases, marker proteins of acquired diseases, marker proteins of cancers, etc. The marker proteins are proteins which are commonly or typically used for diagnosing of related diseases, such as nucleocapsid protein for diagnosing infection of coronavirus; human troponin I and human troponin for diagnosing of acute myocardial infarction and other heart failures; Tau protein for diagnosing of certain neurodegenerative diseases. The target proteins and antibodies which can be detected using the ELISA-PCT test are listed in Table 6 below.

TABLE 6
Target proteins and antibodies for ELISA-PCR test

| | Pair # | Cat# | Description | Source | Clone |
|---|---|---|---|---|---|
| cTnI | Pair 1 | BRNCTNIN113 | cTnI antibody | CHO | 23C12 |
| | | BRJCTNIS108 | cTnI antibody | CHO | 31F1 |
| | Pair 2 | BRNCTNIN113 | cTnI antibody | CHO | 23C12 |
| | | BRNCTNIN103 | cTnI antibody | CHO | 23A3 |
| | | BRNCTNIN110 | cTnI antibody | CHO | 40A10 |
| | Pair 3 | BRNCTNIN113 | cTnI antibody | CHO | 23C12 |
| | | BRNCTNIN103 | cTnI antibody | CHO | 23A3 |
| | | BRJCTNIS108 | cTnI antibody | CHO | 31F1 |
| | — | GRNCTNIN101 | cTnI antigen | E. coli | — |
| cTnT | Pair 1 | BRNCTNTS101 | cTnT antibody | Mouse | 5G8 |
| | | BRNCTNTS102 | cTnT antibody | Mouse | 15C9 |
| Tau | Pair 1 | MAB3494 | Tau antibody | Mouse | 376720 |
| | | MAB106291 | Tau antibody | Mouse | 1032501 |
| Tau | Pair 1 | MN1050 | Tau Thr181 antibody | Mouse | AT270 |
| Thr181 | | MAB106291 | Tau antibody | Mouse | 1032501 |
| CEA | Pair 1 | BECCEAS104 | CEA antibody | CHO | 37C4 |
| | | BENCEAS103 | CEA antibody | Mouse | 3000 |
| | Pair 2 | BENCEAS103 | CEA antibody | Mouse | 3000 |
| | | BECCEAS103 | CEA antibody | Mouse | 1C1 |
| CA199 | Pair 1 | BENCA199S102 | CA199 antibody | CHO | 30C8 |
| | Pair 2 | BENCA199S101 | CA199 antibody | Mouse | 5A9 |
| | | BENCA199S101 | CA199 antibody | Mouse | 5A9 |
| | Pair 3 | BECCA199S103 | CA199 antibody | Mouse | 2A11 |
| | | BENCA199S101 | CA199 antibody | Mouse | 5A9 |
| PSA | Pair 1 | BECPSAS103 | PSA antibody | Mouse | 3D2 |
| | | BENPSAN101 | PSA antibody | Mouse | 3B5 |
| CA153 | Pair 1 | BECCA153S104 | CA153 antibody | CHO | 27B9 |
| | | BEJCA153S108 | CA153 antibody | CHO | 25A3 |
| | Pair 2 | BECCA153S105 | CA153 antibody | CHO | 23F6 |
| | | BEJCA153S108 | CA153 antibody | CHO | 25A3 |

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. An antibody-DNA conjugate set for detection of a target protein, comprising:
a first antibody-DNA conjugate having a first antibody which is configured to bind to the target protein; and a first DNA oligo linked to the first antibody; and
a second antibody-DNA conjugate having a second antibody which is configured to bind to the target protein; and a second DNA oligo linked to the second antibody.

2. The antibody-DNA conjugate set for detection of the target protein according to claim 1, wherein the first DNA oligo is covalently linked to the first antibody, and the second DNA oligo is covalently linked to the second antibody.

3. The antibody-DNA conjugate set for detection of the target protein according to claim 2, wherein the first DNA oligo is covalently linked to the first antibody on its 3′ end, and the second DNA oligo is covalently linked to the second antibody on its 5′ end.

4. The antibody-DNA conjugate set for detection of the target protein according to claim 1, wherein the first DNA oligo and second DNA oligo each has a length between 20-300 nucleotide bases.

5. The antibody-DNA conjugate set for detection of the target protein according to claim 1, wherein the first antibody and second antibody each binds to a different site of the target protein.

6. The antibody-DNA conjugate set for detection of the target protein according to claim 3, wherein when the first antibody and second antibody each binds to the target protein, the 5′ end of the first DNA oligo and the 3′ end of the second DNA oligo attaches to each other.

7. The antibody-DNA conjugate set for detection of the target protein according to claim 6, wherein the first antibody and second antibody, the first DNA oligo and second DNA oligo, and the target protein form a close loop structure.

8. The antibody-DNA conjugate set for detection of the target protein according to claim 1, wherein the target protein is selected from the group consisting of marker proteins of pathogens, marker proteins of infectious diseases; marker proteins of genetic diseases, marker proteins of acquired diseases.

9. The antibody-DNA conjugate set for detection of the target protein according to claim 8, wherein the target protein is a nucleocapsid protein of SARS-Cov2 virus.

10. The antibody-DNA conjugate set for detection of the target protein according to claim 9, wherein the target protein is Troponin I, AZ biomarkers, and cancer biomarkers.

11. A method for quantitatively detecting a target protein with a pair of antibody-DNA conjugates, comprising:
adding a first antibody-DNA conjugate to a sample having the target protein; and,
adding a second antibody-DNA conjugate to the sample having the target protein.

12. The method for quantitatively detecting the target protein according to claim 11, wherein
the first antibody-DNA conjugate having a first antibody which is configured to bind to the target protein and a first DNA oligo linked to the first antibody;
the second antibody-DNA conjugate having a second antibody which is configured to bind to the target protein; and a second DNA oligo linked to the second antibody.

13. The method for quantitatively detecting the target protein according to claim 11, wherein the first DNA oligo is covalently linked to the first antibody on its 3′ end, and the second DNA oligo is covalently linked to the second antibody on its 5′ end.

14. The method for quantitatively detecting the target protein according to claim 13, wherein the first antibody and second antibody each binds to the target protein, the 5′ end of the first DNA oligo and the 3′ end of the second DNA oligo attaches to each other.

15. The method for quantitatively detecting the target protein according to claim 14 further comprising
adding a first primer and a second primer to the sample, wherein the first primer is complementary to a sequence at the 3′ end of the first DNA oligo, and the second primer is identical to a sequence at the 5′ end of the second DNA oligo.

16. The method for quantitatively detecting the target protein according to claim 15 further comprising
starting PCR cycle so as to amplify the first and second DNA oligos with the first and second primers.

17. The method for quantitatively detecting the target protein according to claim 16 further comprising
quantitatively analyzing the result of the PCR amplification so as to determine the amount of the target protein in the sample.

18. The method for quantitatively detecting the target protein according to claim 11, wherein the target protein is selected from the group consisting of marker proteins of pathogens, marker proteins of infectious diseases; marker proteins of genetic diseases, marker proteins of acquired diseases.

19. The method for quantitatively detecting the target protein according to claim 18, wherein the target protein is a nucleocapsid protein of SARS-Cov2 virus.

20. The method for quantitatively detecting the target protein according to claim 19, wherein the target protein is Troponin I.


SEQUENCE LISTING (80 sequences)

SEQ ID NO: 1 - DNA, 60 bp, synthetic construct
  source 1..60: mol_type = other DNA
aattggacgtggctttcgcgttagtacgtagcatggtcacacaagcacagtagatcctgc

SEQ ID NO: 2 - DNA, 60 bp, synthetic construct
  source 1..60: mol_type = other DNA
tcataaggagtccggtgtagcgaaagatcaaggcgaccctaggtagcaaccgccggctcc

SEQ ID NO: 3 - DNA, 60 bp, synthetic construct
  source 1..60: mol_type = other DNA
cttccgctgggatccaacgttggcggccgaagccgccattccatagtgagtccttcgtct

SEQ ID NO: 4 - DNA, 60 bp, synthetic construct
  source 1..60: mol_type = other DNA
tcctagataccgcactctgggcagtacgaggtaatgccagtcacccagtgccgaacaaca

SEQ ID NO: 5 - DNA, 60 bp, synthetic construct
  source 1..60: mol_type = other DNA
tccagttcggtcagtgggtcactgcaagtagtcgattgcattgccaatctccgagtgatt

SEQ ID NO: 6 - DNA, 60 bp, synthetic construct
  source 1..60: mol_type = other DNA
aaaatcaccagtgcccaagaccaggggggctcgccgcgttggctaatcccggtacatctt

SEQ ID NO: 7 - DNA, 60 bp, synthetic construct
  source 1..60: mol_type = other DNA
cgagcggcgcagccgattaggaccatgtagaacatttgttacaagacttcttttaaacac

SEQ ID NO: 8 - DNA, 60 bp, synthetic construct
  source 1..60: mol_type = other DNA
gcttagttcaacctcgaatacctcgtatcattgtgcacctgccggtcaccagccaacgat

SEQ ID NO: 9 - DNA, 60 bp, synthetic construct
  source 1..60: mol_type = other DNA
atgacatgtggatgggcagtggccggttgttacacgcctgccgcgacgctgaatgacccg

SEQ ID NO: 10 - DNA, 60 bp, synthetic construct
  source 1..60: mol_type = other DNA
cggtctagctgactgtctatcgcctaggtcaaatagggagctttgatatctgcgtgtcca

SEQ ID NO: 11 - DNA, 60 bp, synthetic construct
  source 1..60: mol_type = other DNA
agtttatcccaccaaactatagccgtacaggccgaaatcttaagtcatatcgcgcgacta

SEQ ID NO: 12 - DNA, 60 bp, synthetic construct
  source 1..60: mol_type = other DNA
gcgtgcccagggtatattaggtcagcatcggatggactgacatgaacctttacaccgaag

SEQ ID NO: 13 - DNA, 60 bp, synthetic construct
  source 1..60: mol_type = other DNA
gtccacccgaccgtacatagaaatgagggtccccgtacgcccacgcacctgttcgctcgt

SEQ ID NO: 14 - DNA, 60 bp, synthetic construct
  source 1..60: mol_type = other DNA
gtcaccatgtaccaagggcgataacgatcggtgggagtattcatcgtggtgaagacgctg

SEQ ID NO: 15 - DNA, 60 bp, synthetic construct
  source 1..60: mol_type = other DNA
gccggcgtggaaggtaacagcaccgctgcgagcctaatgcgccgtttccacgaacacagg

SEQ ID NO: 16 - DNA, 60 bp, synthetic construct
  source 1..60: mol_type = other DNA
tcctggcctgcttgatgtctcgtgaccttcttagagatggacgaaatgtttcgcgaccta

SEQ ID NO: 17 - DNA, 60 bp, synthetic construct
  source 1..60: mol_type = other DNA
aggacctctagctcctttacaaagtgctggttccctttccggcgggatgccttatctaaa

SEQ ID NO: 18 - DNA, 60 bp, synthetic construct
  source 1..60: mol_type = other DNA
gtataatgctggagccctcccccaagcgttcagggtggggtttgctacgacttccgagtc

SEQ ID NO: 19 - DNA, 60 bp, synthetic construct
  source 1..60: mol_type = other DNA
aagtggcagcctaaacgatgttgggggctcgcgatgcacacgctctggtacaatacatac

SEQ ID NO: 20 - DNA, 60 bp, synthetic construct
  source 1..60: mol_type = other DNA
caaaagctttaaacgcgagttcccgcccataacctggaccgaatgcgggatcatgcatcg

SEQ ID NO: 21 - DNA, 40 bp, synthetic construct
  source 1..40: mol_type = other DNA
ccgcgcatcctatgtatcaagttagttctaatggaatacg

SEQ ID NO: 22 - DNA, 40 bp, synthetic construct
  source 1..40: mol_type = other DNA
ggcggtaaggtatcactcaagaagcaggctcggtaagaca

SEQ ID NO: 23 - DNA, 40 bp, synthetic construct
  source 1..40: mol_type = other DNA
gtgactatctgtgccagatcgtctggcagactgccgatcc

SEQ ID NO: 24 - DNA, 40 bp, synthetic construct
  source 1..40: mol_type = other DNA
cctgacctaacggtaagaggctcacataatggctccgccg

SEQ ID NO: 25 - DNA, 40 bp, synthetic construct
  source 1..40: mol_type = other DNA
tagcgtgacggccgcaggggacccataaaatgcgatcgta

SEQ ID NO: 26 - DNA, 40 bp, synthetic construct
  source 1..40: mol_type = other DNA
gtaatgaatattcagtagaaagtctgtgttagagggacga

SEQ ID NO: 27 - DNA, 40 bp, synthetic construct
  source 1..40: mol_type = other DNA
tatcttcctgcccagtggcggatggttatcgctgttgcta

SEQ ID NO: 28 - DNA, 40 bp, synthetic construct
  source 1..40: mol_type = other DNA
gtgcggacggcgttgcaacttccagggcctaatctgaccg

SEQ ID NO: 29 - DNA, 40 bp, synthetic construct
  source 1..40: mol_type = other DNA
gactagagtggcgagaactatggcgtgtgacccgttatgc

SEQ ID NO: 30 - DNA, 40 bp, synthetic construct
  source 1..40: mol_type = other DNA
gccttaggattcacttcagcgcgcaggcctgggtcgagat

SEQ ID NO: 31 - DNA, 40 bp, synthetic construct
  source 1..40: mol_type = other DNA
ggctcagctctatttttgtggtcatgggttctggtccgcc

SEQ ID NO: 32 - DNA, 40 bp, synthetic construct
  source 1..40: mol_type = other DNA
cggaaacgggtgcgtggactagcgaggagcaaacgaaaat

SEQ ID NO: 33 - DNA, 40 bp, synthetic construct
  source 1..40: mol_type = other DNA
cgtttgcatttaagggccgcacgaaccacagagcataagg

SEQ ID NO: 34 - DNA, 40 bp, synthetic construct
  source 1..40: mol_type = other DNA
ggtttacgtgggaaaggtgcttgtgtcccaacaggctagg

SEQ ID NO: 35 - DNA, 40 bp, synthetic construct
  source 1..40: mol_type = other DNA
gctgtccgatcgtatattaggactccgcgatggggttggc

SEQ ID NO: 36 - DNA, 40 bp, synthetic construct
  source 1..40: mol_type = other DNA
ggaagaggtcgccctacaaaatagatttgcgctactctcc

SEQ ID NO: 37 - DNA, 40 bp, synthetic construct
  source 1..40: mol_type = other DNA
cgcaatgacggacgtattcctctggccacatcggttcctg

SEQ ID NO: 38 - DNA, 40 bp, synthetic construct
  source 1..40: mol_type = other DNA
caacgtgtccgtgttcacgttatatgcgcacaaggccgag

SEQ ID NO: 39 - DNA, 40 bp, synthetic construct
  source 1..40: mol_type = other DNA
gtgttccggctgttatcctgcatcggaacgtcaatcatgc

SEQ ID NO: 40 - DNA, 40 bp, synthetic construct
  source 1..40: mol_type = other DNA
ttccactgtgtttgtctcatgtaggacgggcgcaaagcgt

SEQ ID NO: 41 - DNA, 25 bp, synthetic construct
  source 1..25: mol_type = other DNA
aattggacgtggctttcgcgttagt

SEQ ID NO: 42 - DNA, 25 bp, synthetic construct
  source 1..25: mol_type = other DNA
tcataaggagtccggtgtagcgaaa

SEQ ID NO: 43 - DNA, 25 bp, synthetic construct
  source 1..25: mol_type = other DNA
cttccgctgggatccaacgttggcg

SEQ ID NO: 44 - DNA, 25 bp, synthetic construct
  source 1..25: mol_type = other DNA
tcctagataccgcactctgggcagt

SEQ ID NO: 45 - DNA, 25 bp, synthetic construct
  source 1..25: mol_type = other DNA
tccagttcggtcagtgggtcactgc

SEQ ID NO: 46 - DNA, 25 bp, synthetic construct
  source 1..25: mol_type = other DNA
aaaatcaccagtgcccaagaccagg

SEQ ID NO: 47 - DNA, 25 bp, synthetic construct
  source 1..25: mol_type = other DNA
cgagcggcgcagccgattaggacca

SEQ ID NO: 48 - DNA, 25 bp, synthetic construct
  source 1..25: mol_type = other DNA
gcttagttcaacctcgaatacctcg

SEQ ID NO: 49 - DNA, 25 bp, synthetic construct
  source 1..25: mol_type = other DNA
atgacatgtggatgggcagtggccg

SEQ ID NO: 50 - DNA, 25 bp, synthetic construct
  source 1..25: mol_type = other DNA
cggtctagctgactgtctatcgcct

SEQ ID NO: 51 - DNA, 25 bp, synthetic construct
  source 1..25: mol_type = other DNA
agtttatcccaccaaactatagccg

SEQ ID NO: 52 - DNA, 25 bp, synthetic construct
  source 1..25: mol_type = other DNA
gcgtgcccagggtatattaggtcag

SEQ ID NO: 53 - DNA, 25 bp, synthetic construct
  source 1..25: mol_type = other DNA
gtccacccgaccgtacatagaaatg

SEQ ID NO: 54 - DNA, 25 bp, synthetic construct
  source 1..25: mol_type = other DNA
gtcaccatgtaccaagggcgataac

SEQ ID NO: 55 - DNA, 25 bp, synthetic construct
  source 1..25: mol_type = other DNA
gccggcgtggaaggtaacagcaccg

SEQ ID NO: 56 - DNA, 25 bp, synthetic construct
  source 1..25: mol_type = other DNA
tcctggcctgcttgatgtctcgtga

SEQ ID NO: 57 - DNA, 25 bp, synthetic construct
  source 1..25: mol_type = other DNA
aggacctctagctcctttacaaagt

SEQ ID NO: 58 - DNA, 25 bp, synthetic construct
  source 1..25: mol_type = other DNA
gtataatgctggagccctcccccaa

SEQ ID NO: 59 - DNA, 25 bp, synthetic construct
  source 1..25: mol_type = other DNA
aagtggcagcctaaacgatgttggg

SEQ ID NO: 60 - DNA, 25 bp, synthetic construct
  source 1..25: mol_type = other DNA
caaaagctttaaacgcgagttcccg

SEQ ID NO: 61 - DNA, 25 bp, synthetic construct
  source 1..25: mol_type = other DNA
cgtattccattagaactaacttgat

SEQ ID NO: 62 - DNA, 25 bp, synthetic construct
  source 1..25: mol_type = other DNA
tgtcttaccgagcctgcttcttgag

SEQ ID NO: 63 - DNA, 25 bp, synthetic construct
  source 1..25: mol_type = other DNA
ggatcggcagtctgccagacgatct

SEQ ID NO: 64 - DNA, 25 bp, synthetic construct
  source 1..25: mol_type = other DNA
cggcggagccattatgtgagcctct

SEQ ID NO: 65 - DNA, 25 bp, synthetic construct
  source 1..25: mol_type = other DNA
tacgatcgcattttatgggtcccct

SEQ ID NO: 66 - DNA, 25 bp, synthetic construct
  source 1..25: mol_type = other DNA
tcgtccctctaacacagactttcta

SEQ ID NO: 67 - DNA, 25 bp, synthetic construct
  source 1..25: mol_type = other DNA
tagcaacagcgataaccatccgcca

SEQ ID NO: 68 - DNA, 25 bp, synthetic construct
  source 1..25: mol_type = other DNA
cggtcagattaggccctggaagttg

SEQ ID NO: 69 - DNA, 24 bp, synthetic construct
  source 1..24: mol_type = other DNA
gcataacgggaggccctggtagtt

SEQ ID NO: 70 - DNA, 24 bp, synthetic construct
  source 1..24: mol_type = other DNA
atctcgaccaggccctggcgctga

SEQ ID NO: 71 - DNA, 24 bp, synthetic construct
  source 1..24: mol_type = other DNA
ggcggaccagaggccctggcacaa

SEQ ID NO: 72 - DNA, 24 bp, synthetic construct
  source 1..24: mol_type = other DNA
attttcgtttaggccctggagtcc

SEQ ID NO: 73 - DNA, 24 bp, synthetic construct
  source 1..24: mol_type = other DNA
ccttatgctcaggccctgggcggc

SEQ ID NO: 74 - DNA, 24 bp, synthetic construct
  source 1..24: mol_type = other DNA
cctagcctgtaggccctgggcacc

SEQ ID NO: 75 - DNA, 25 bp, synthetic construct
  source 1..25: mol_type = other DNA
gccaaccccaaggccctggcctaat

SEQ ID NO: 76 - DNA, 24 bp, synthetic construct
  source 1..24: mol_type = other DNA
ggagagtagcaggccctggtttgt

SEQ ID NO: 77 - DNA, 24 bp, synthetic construct
  source 1..24: mol_type = other DNA
caggaaccgaggccctggaggaat

SEQ ID NO: 78 - DNA, 24 bp, synthetic construct
  source 1..24: mol_type = other DNA
ctcggccttgaggccctggacgtg

SEQ ID NO: 79 - DNA, 24 bp, synthetic construct
  source 1..24: mol_type = other DNA
gcatgattgaaggccctggcagga

SEQ ID NO: 80 - DNA, 24 bp, synthetic construct
  source 1..24: mol_type = other DNA
acgctttgcgaggccctggatgag